(19) United States
(12) Patent Application Publication
(10) Pub. No.: US 2010/0081208 A1
(43) Pub. Date: Apr. 1, 2010

(54) Multirange indicator

(75) Inventors: Klaus KEITE-TELGENBÜSCHER, Hamburg (DE); Hermann NEUHAUS-STEINMETZ, Ahrensburg (DE)

Correspondence Address: Briscoe, Kurt G.;Norris McLaughlin & Marcus, PA, 875 Third Avenue, 8th Floor, New York, NY, 10022, US

(73) Assignee: Tesa SE, Hamburg (DE)

(21) Appl. No.: 12/565,961

(22) Filed: Sep. 24, 2009

(30) Foreign Application Priority Data
Oct. 1, 2008 (DE) 10 2008 049 848.3

(51) Int. Cl. G01N 21/78; C07C 381/04; C07C 45/00; C07C 315/00; C07C 255/46

(52) U.S. Cl. 436/164; 568/77; 568/300; 568/28; 558/388; 568/959

(57) ABSTRACT

A multirange indicator is proposed which permits quantitative dose determination of high-energy actinic radiation, where the dose for different wavelength ranges can be respectively determined in parallel. To this end, the multirange indicator has two indicator systems designed with mutually corresponding properties, thus eliminating any mutual distortion of the results of the dose measurements. The first indicator system is based on a photolatent Lewis acid or photolatent Lewis base, and the second indicator system is based on a polysubstituted triphenylmethane dye. Both the multirange indicator and its use for the production of various dose-measurement devices are described. Finally, the following are proposed for the measurement of UV radiation and/or electron-beam radiation: as dose-measurement device, a dose-measurement coating material and a flat dose-measurement element, these being based on the above multirange indicator, and also the measurement method that can be implemented with the said dose-measurement devices.

The invention relates to a multirange indicator with a first indicator system, which encompasses a first indicator dye and encompasses a photolatent Lewis acid or photolatent Lewis base, and which, on exposure of the photolatent Lewis acid or, respectively, photolatent Lewis base to electromagnetic radiation with wavelengths from a first radiation wavelength range, is capable of liberating a Lewis acid or, respectively, Lewis base and of changing the light absorption of the first indicator dye during a first reaction of the first indicator dye with the liberated Lewis acid or, respectively, Lewis base. The invention further relates to the use of the abovementioned multirange indicator for the production of a dose-measurement coating for the measurement of UV radiation and/or electron-beam radiation, to the resultant flat dose-measurement element for the measurement of UV radiation and/or electron-beam radiation, encompassing a radiation-sensitive layer with the abovementioned multirange indicator, and also to a method for the determination of a dose of electromagnetic radiation incident on a test specimen.

During a chemical reaction, structural changes occur to one or more reactants. By way of example, in a photochemical reaction, as long as the wavelength and intensity of the electromagnetic radiation have been selected in such a way that the radiation is absorbed by an agent (one of the reactants), the interaction of the agent with electromagnetic radiation causes an alteration in the molecular nature of the agent.

Since exposure to the radiation on the occasion of a photochemical reaction causes an “action”, electromagnetic radiation with a wavelength at which a reaction is initiated is also termed “actinic radiation”. Actinic radiation therefore involves, for example, high-energy light, an example being radiation from the ultraviolet region of the spectrum (UV radiation, UV light), or blue light (actinic light). Examples of other forms of actinic radiation are gamma rays or electron beams, for example anode rays.

DIN 5031, Part 7 divides the wavelengths of radiation in the ultraviolet region into three subregions, namely into the UVA, UVB and UVC regions. The wavelengths of ultraviolet radiation from the UVA region are from 380 nm to 315 nm (known as “black light”), this therefore being low-energy UV radiation. The wavelengths of ultraviolet radiation from the UVB region are from 315 nm to 280 nm, while the wavelengths of ultraviolet radiation from the UVC region, with the highest energy level, are from 280 nm to 100 nm. There are also other conventional subdivisions used in practice alongside this classification system, examples being subdivision into near UV (NUV; wavelength: 400 nm-200 nm), far UV (also termed “vacuum UV”; FUV, VUV; wavelength: 200 nm-10 nm) and extreme UV (EUV, XUV; wavelength: 31 nm-1 nm).

Radiation with a wavelength at which the agent exhibits no absorption cannot interact with the agent and cannot therefore cause any photochemical reaction. In order that a photochemical reaction can take place at all, the wavelength of the actinic radiation used must therefore have at least some overlap with the spectral absorption of the agent. By way of example, molecular reactants usually have absorption bands which in spectral terms are very narrow, and it is mostly difficult to find radiation sources with an emission characteristic specifically matched to the absorption of such a reactant. Radiation sources which are therefore often used for photochemical reactions are actinic sources which emit actinic radiation within a broad spectral range, where only a small portion of this radiation is absorbed by the agent.

The conversion obtained in a photochemical reaction can depend inter alia on the total number of incident photons having the correct energy level, and therefore on the dose of actinic radiation. The radiation dose is in turn a function of the irradiation intensity and the irradiation time. Monitoring of the conversion actually achieved in a reaction therefore requires that the progress of a photochemical reaction of an agent be monitored in situ, for example when a polymer system is hardened during irradiation with UV light. Only a portion of the actinic radiation emitted has a wavelength which is absorbed by the agent, and it is therefore desirable, for exact monitoring of the incident dose, that the method used to determine the amount of actinic radiation is such as to detect the different wavelength ranges separately, and thus to permit determination of the actual relevant energy dose.

An additional factor here is that the overall performance of conventional devices for producing actinic radiation, and also their spectral intensity distribution, can change during their operation. By way of example, electrically operated UV radiation sources conventional in industry lose about one fifth of their initial intensity during the first thousand hours of operation, and are moreover extremely sensitive to contamination. An essential requirement for reliable monitoring of a photochemical reaction used in industry is therefore at least some extent of spectral resolution in detection of the actinic radiation, so that chronological changes in the intensity of irradiation are recorded, thus making it simpler to analyse causes of variations in product quality.

There are existing electronic dose-measurement devices for this purpose, permitting range-differentiation in determining the dose of incident UV light. By way of example, the electronic “Power Puck” UV-dose-measurement device from the company EIT (USA) can measure doses in four different wavelength ranges, namely in the ranges from 250 nm to 260 nm, from 280 nm to 320 nm, from 320 nm to 390 nm and from 395 nm to 445 nm.

However, a disadvantage of electronic dose-measurement devices is that their dimensions are relatively large. By way of example, the diameter of the “Power Puck” dose-measurement device is about 100 mm and its height is about 15 mm. The dimensions of devices of this type often make them unsuitable for monitoring exposure to radiation of products within closed manufacturing lines and processing plants.

If, for example, the intention is to monitor a crosslinking reaction of web-type polymer layers, where the reaction takes place with exposure to ultraviolet radiation (in the case of what are known as “radiation-crosslinking polymer systems”), it is impossible to use electronic dose-measurement devices because the web-type material is conducted past UV lamps generally arranged in the immediate vicinity of deflector rolls (i.e. directly in front of/behind or above/under these), where there is insufficient space available for the positioning of the dose-measurement device on the web-type material.

A consequence of the height of electronic dose-measurement devices is moreover that the radiation dose is not recorded directly at the location of the web-type material, but instead slightly above the said location (about 15 mm above in the case of “Power Puck”). The measured values obtained are therefore not a true reflection of the radiation dose in the irradiation plane, but instead are distorted values, and this can lead to considerable deviations from the actual radiation dose at the location of the product, in particular in the case of oblique incidence of radiation, when the geometry of the measurement detector is taken into account.

The situation is even more problematic if the intention is to determine the UV radiation dose for three-dimensional mouldings rather than for web-type materials, for example in order to ensure homogeneous crosslinking of radiation-crosslinked mouldings. In this case, correct recording requires that the radiation dose be determined precisely at the location of the respective surface regions of the mouldings, and it is impossible to achieve this in practice with an electronic dose-measurement device.

It is therefore often impossible, when monitoring the processing of photochemically reacting systems, to determine the actual radiation dose reliably by means of electronic dose-measurement devices, the result being that, instead of this, complicated quality assurance has to be carried out via monitoring of a large number of different product features.

As an alternative to electronic measurement, it is also possible to use a chemical method of determining radiation dose, by adding, to the agent, an indicator substance which provides an optically detectable indication of the respective conversion level. The indicator used usually comprises a system in which a further photochemical reaction proceeds on exposure to the respective actinic light, and this reaction causes a change in the absorption of the indicator. The extent of colour change can be used to follow the respective radiation dose qualitatively and sometimes also quantitatively, thus permitting determination of the actual conversion of the agent. This monitoring can use visual methods or an apparatus.

Examples of the use of indicator systems in which exposure to actinic radiation produces a colour change (known as “radiochromic systems”), are measurement of the incident dose of actinic radiation or identification of substrate regions exposed to the light, for example when photoresist plates are exposed to light.

The prior art discloses a large number of liquid or solid systems which can provide visual detection of exposure to actinic radiation. By way of example, U.S. Pat. No. 4,923,781 describes photopolymerizable image-generating systems, and EP 0 291 880 B1 describes photopolymerizable recording materials, while U.S. Pat. No. 5,677,107 discloses photochemically engendered three-dimensional shaping processes.

Application-specific dose-measurement systems (known as “dosimeters”) based on photochemical reactions are inter alia the subject matter of U.S. Pat. No. 4,130,760, U.S. Pat. No. 3,787,687 and DE 197 19 721 C1. The systems proposed in the said documents respectively have an indicator dye, which absorbs the actinic radiation and as a consequence of the energy thus absorbed reacts with alteration of its absorption, thus then permitting determination of the absorbed dose via colour comparison, if appropriate after a further step for the development of the detectable dye.

However, there are only a few known compounds which simultaneously exhibit absorption in the wavelength range of detectable actinic radiation (detectable wavelength) and photochemically influenceable absorption in the wavelength range of visible light (observation wavelength or indication wavelength) for the visual indication of the absorbed dose. The number of direct-reaction indicator systems is therefore relatively small, and only a few wavelength ranges are therefore accessible for direct detection of actinic radiation.

In contrast, indirect-reaction indicator systems have the advantage that they can be adapted as required and can thus serve to detect actinic radiation from various wavelength ranges. An indirect-reaction indicator system is an indicator system which comprises a photochemically active substance and also comprises an indicator dye. On irradiation with actinic radiation, the incident radiation is absorbed by the photochemically active substance. A reaction of the photochemically active substance takes place as a consequence of the energy thus absorbed. The intermediate agents (or intermediates) produced during the reaction in turn react with the indicator dye, changing its absorption in the visible region of the light spectrum. Although it is possible during this type of indirect reaction that a change in the absorption of the photochemically active substance additionally takes place in the visible region of the spectrum, any possible occurrence of an effect of this type is usually insignificant.

In the case of indirect-reaction systems, the substances which absorb in the wavelength range of the actinic radiation to be detected, and the substances whose absorption changes in the wavelength range of visible light can be selected differently, and the detection wavelength and the indication wavelength have therefore been decoupled from one another, and the photochemically active substance and the indicator substance can therefore easily be selected appropriately for the respective experimental conditions and adapted to these conditions within a wide range.

There are some recently disclosed examples of indirect-reaction indicator systems which comprise, as photochemically active substance, a photolatent acid (an acid generator). This photolatent acid is not present as acid in the unirradiated state, but liberates an acid only on irradiation with actinic radiation. The liberated acid reacts with an indicator dye which is colourless in the neutral region and which is used as colour generator (latent dye; dye precursor; an example being a leuco dye), and which changes its absorption in the visible region of the light spectrum as a consequence of the chemical reaction with the liberated acid. The intensity of the colour change here depends on the amount of acid liberated photochemically from the photolatent acid. WO 2005/097876 A1 describes various photolatent acids, indicator dyes and matrix systems for absorption of the photolatent acid and of the indicator dye.

The wavelength range (the detection wavelengths) within which this type of system can be used as indicator depends in essence on the absorption of the photolatent acid of the indicator system in the wavelength range of the actinic radiation. The photolatent acid used can comprise a wide variety of different latent acids which respectively are converted into an acid on exposure to actinic radiation, and some of which differ greatly in terms of their absorption in the wavelength range of the actinic radiation.

Accordingly, the said indirect-reaction indicator system comprises a detection-wavelength-specific photolatent acid and a non-detection-wavelength-specific indicator dye (which, however, is indicator-wavelength-specific), and also, as intermediate agent, an acid which is liberated photochemically from the photolatent acid and which reacts with the indicator dye with a change in the colour of the indicator dye. Selection of a photolatent acid with a suitable absorption spectrum gives indicator systems which can be used for specific detection of actinic radiation from almost any wavelength range.

However, a disadvantage of these indirect-reaction indicator systems is that the colour change achieved therewith is not retained permanently, and the extent of colour change is therefore unstable, and the colour of the materials alters within a short time with exposure to light, moisture and/or heat.

Another disadvantageous factor is that the intensity of the colour change achieved with the said systems does not generally have any defined mathematical relationship with the total incident dose, and quantitative determination of the incident dose is therefore possible only after extensive and therefore complicated calibration, if indeed it is possible at all.

These systems have moreover proved to be impractical, because they are often capable of photochemical reaction not only with actinic radiation but also with light of other wavelengths, e.g. visible light, and they therefore generally have poor shelf life in the unirradiated state, and also require very careful handling.

There is therefore a fundamental need for indicators which permit reliable dose determinations in a variety of wavelength ranges; requirements for this are stable colour changes and colour-change intensities which are a defined function of the incident dose.

One of the advantages of the indirect-reaction indicator systems consists in the large number of available photolatent acids which provide absorption at a variety of wavelengths, and which can be used to cover a wide range of detection wavelengths. Indeed, according to WO 2005/097876 A1 it is possible to produce an indicator system with a plurality of photolatent acids whose absorption spectra mutually overlap, thus permitting detection of actinic radiation within a large range of wavelengths. However, the acids liberated from the various photolatent acids react with the dye in an identical manner, in the same colour reaction, and therefore non-specifically. These indicator systems are therefore capable only of integral detection of actinic radiation, without any possibility of distinguishing between individual wavelength ranges.

Accordingly, although it would be possible by using the indicator system known from the prior art to detect the total incident dose of polychromatic actinic radiation, there would be no differentiation here between low-energy UVA radiation and high-energy UVC radiation. These indirect-reaction indicators would at most be capable of detection-wavelength-selective detection if two indicator systems were to be used which absorb in different wavelength ranges and which are moreover spatially separate from one another. However, if the intention is to use chemical indicators to achieve the functionality of conventional electronic dose-measurement devices, it is precisely this type of detection-wavelength-selective detection that is essential.

There is therefore a need for a multirange indicator which provides parallel detection in particular of the dose of actinic radiation with wavelengths from different wavelength ranges, so that it is possible to distinguish with adequate measurement accuracy between at least two wavelength ranges within a single determination.

In principle, a general concept for the production of multirange indicators can consist in using a plurality of indicator systems within a single indicator. However, when this general concept is put into practice, it is found to be extremely difficult to find suitable indicator systems which can be used for the various detection wavelengths and which do not have any effect on one another.

This is particularly problematic in the case of a multirange indicator which has a plurality of indicator systems of which one is based on the use of photolatent acids or bases (or—still more generally—on the use of photolatent Lewis acids or of photolatent Lewis bases), where these react with an indicator dye and thus bring about a colour change, since almost all organic indicator dyes contain chemical groups which can interact with the acids or bases produced as intermediates.

In most cases, the—mostly non-specific—interaction between the Lewis acids or Lewis bases produced as intermediates and the indicator dyes is attributable to the property of the Lewis acids or Lewis bases to act as electron-pair acceptors or electron-pair donors. By way of example, Lewis acids can interact with groups on organic dye molecules, for example groups having oxygen atoms or having nitrogen atoms, e.g. with ether, hydroxy, carbonyl or amino groups, a possible result being formation of Lewis adducts (Lewis-acid-Lewis-base reaction).

If the Lewis acid or Lewis base of a first indicator system interacts with the indicator dye of another indicator system, the entire amount of the Lewis acids produced is not available to contribute to a colour change of the first indicator system. Accordingly, the result obtained from a quantitative dose determination using the first indicator system is lower than the result that would correspond to the actual incident dose.

An even more problematic situation arises if the reaction of the indicator dye of the second indicator system with the Lewis acid or Lewis base also causes a colour change in the said indicator system, since this distorts the quantitative determination provided by both indicator systems, in that the determination provided by the first indicator system gives an excessively low value and the determination provided by the second indicator system in contrast gives an excessively high value, because of the additional contribution.

It is not even possible here to make a subsequent correction to remove the distortion between the systems, since the only way to achieve this would be to have knowledge of the absolute intensities of the radiation to be detected for both wavelength ranges; however, the multirange indicator should be used precisely with the intention of determining these intensities. An additional factor is that the molar extinction coefficients of the dyes are different, and it is therefore not possible simply to convert a change in absorption at the indicator wavelength of one of the indicator dyes to a change in concentration of the other indicator dye.

Accordingly, the specific object of the present invention consisted in providing a reliable multirange indicator with an indicator system based on a first indicator dye and on a photolatent Lewis acid or photolatent Lewis base, where there is additionally a second indicator system provided whose level of interaction with the first indicator system is sufficiently small as to permit quantitative dose determinations in parallel at the different detection wavelengths. With regard to the reliability of dose determination here, the requirement is respectively a stable colour change, and also, for the different wavelength ranges, a colour-change intensity which is a function of the incident dose.

This object is achieved according to the invention by a multirange indicator of the type mentioned in the introduction, in which, in addition to the first indicator system, there is a second indicator system present which encompasses a second indicator dye, and which is capable of changing the light absorption of the second indicator dye in a second reaction on exposure to electromagnetic radiation with wavelengths from a second radiation wavelength range, where the second indicator dye is a triphenylmethane dye, in which the central carbon atom of the methane group has bonding to a structural unit selected from the group consisting of halogens, pseudohalogens, chalcogens, sulphates and substituted or unsubstituted tosylates, and in which at least one of the C₆ rings of the phenyl groups has, in ortho- or para-position with respect to the bond to the central methyl carbon atom, an electron-withdrawing substituent with +M effect, and/or has, in meta-position with respect to the bond to the central methyl carbon atom, an electron-donating substituent with +M effect.

The use of two photochromic colour-change systems whose spectral sensitivity ranges (absorption) at least do not completely overlap with one another, for simultaneous dose determination in at least two different wavelength ranges is in principle an achievable object. However, determination of the dose of actinic radiation for two different wavelength ranges is possible only if the radiation wavelength ranges of the first reaction and of the second reaction are different. Furthermore, if dose determination is to be carried out for the two different wavelength ranges independently of one another and in parallel alongside one another, it is essential to use two indicator systems which differ in respect of the mechanism of the colour-change reaction of the indicator dyes.

The finding, which is surprising and was not foreseeable by the person skilled in the art, is that if, in addition to an indicator system with a first indicator dye and with a photolatent Lewis acid or photolatent Lewis base, a second indicator system is used which comprises, as indicator dye, a compound from the particular class of dyes described above, the resultant multirange indicator permits reliable parallel determination of the radiation doses in different wavelength ranges, where the indicator systems present therein do not distort the results of determination but instead, independently of one another, permit exact determination of the incident dose.

This is all the more astounding because indicator dyes which contain structural units having non-bonding electron pairs (i.e. halogens, pseudohalogens, chalcogens, sulphates and tosylates, and also other substituents having a +M effect) and which moreover also have electron-withdrawing or electron-donating substituents having additional electron pairs should give rise to particularly high levels of interaction in particular with Lewis acids, and accordingly severe distortion of the results of determination would also be expected; however, this has not been observed in the present case.

It has proved advantageous here to design the multirange indicator as multirange UV indicator and to adapt it in such a way that the electromagnetic radiation absorbed by the first indicator system and the electromagnetic radiation absorbed by the second indicator system is respectively light from the ultraviolet region of the spectrum. A multirange indicator is thus obtained which is particularly suitable for that type of actinic radiation which is most frequently used in photoreaction technology.

In particular, it is advantageous here if one of the indicator systems is capable of absorbing light from the UVA region of the spectrum and the other indicator system is capable of absorbing light from the UVB region and/or from the UVC region of the spectrum, for example in that the first indicator system is capable of absorbing UVA radiation and the second indicator system is capable of absorbing UVB and/or UVC radiation. It is thus possible to detect the relatively high-energy UVB and UVC radiation, which is only of low intensity in the emission spectrum of conventional UV sources, independently of the low-energy UVA radiation, which has higher intensity in the emission spectrum of conventional UV sources. It is thus possible to select the concentrations of the indicator systems respectively in such a way that the multirange indicator has, in both wavelength ranges, a sensitivity adapted to the emission spectrum of the UV source, thus providing ideal utilization of the measurement ranges and therefore improved sensitivity. This effect can be additionally improved by using inert absorbent filter materials for the controlled attenuation of the UV light from the different wavelength ranges, and these can by way of example be arranged within the multirange indicator or as a separate filter foil.

It has also proved advantageous if the first indicator dye and/or the second indicator dye take(s) the form of leuco dye prior to exposure to the electromagnetic radiation (in the storage state), thus ensuring that the colour-change reaction has particularly good detectability, with maximum brightness contrast, which can also be perceived qualitatively by the naked eye, namely a colour change from colourless to coloured.

With regard to the first indicator system, it is advantageous if its photolatent Lewis acid encompasses at least one photolatent Brønsted acid, and its first indicator dye encompasses at least one Brønsted-acid-sensitive dye. This permits rapid progress of the colour-change reaction and therefore almost immediate determination of total dose. At the same time, it is possible to select the photolatent Lewis acid involved here from a wide variety of known latent acids and thus to achieve almost any desired selection of the spectral position and width of the range of detection wavelength for the first indicator system. Use of this type of first indicator system moreover gives a particularly low level of interaction with the second indicator system to be used according to the invention.

In particular, it is advantageous to use a fluoran as acid-sensitive dye, because this method can give a rapid colour change, where the colour changes, over a large dose range, with the incident dose in a manner which is defined and therefore calculable, and has particularly high colour stability, and also thermal stability.

In contrast, for the second indicator system it is useful if its second indicator dye is a triphenylmethane dye whose structural unit bonded to the central carbon atom of the methane group is a cyano group. This design can give a colour change which proceeds rapidly via heterolytic cleavage of the nitrile group in the form of cyanide anion, and removal of the resultant cyanide anion from the equilibrium for example via complexing with transition metal cations present in the indicator, so that the colour change is very stable with respect to visible light and with respect to temperature increases. It is preferable here to use pararosaniline nitrile as triphenylmethane dye, since this dye can achieve a particularly marked colour change, the intensity of which changes in a mathematically defined manner with the incident dose over a large dose range and is moreover stable.

A multirange indicator which has proven to be very particularly advantageous is one in which the photolatent Lewis acid of the first indicator system encompasses at least one photolatent acid (namely a Brønsted acid) and the first indicator dye of the first indicator system encompasses at least one acid-sensitive dye from the group of the fluorans, and in which the second indicator dye is pararosaniline nitrile. Specifically when this particular combination of the first indicator system and of the second indicator system is used, it has been found that, in addition to the advantageous properties described above, the two indicator systems do not have any adverse effect on one another and moreover the use of the indicator system with a fluoran and with a latent acid significantly improves the long-term stability of the colour change of the pararosaniline nitrile. The achievable stability of the colour change of the pararosaniline nitrile dye here was not only considerably greater than in indicator mixtures which respectively comprised only one of the two indicator systems; it was moreover also possible to omit the use of further stabilizing auxiliaries in the indicator systems, without any resultant reduction in the stability of the colour change.

It is moreover advisable that the amount present of the first indicator system and/or of the second indicator system in the multirange indicator is respectively at least 0.01% by weight and at most 10% by weight. This type of content firstly ensures that the colour change that occurs in the indicator dyes is easy to detect, and secondly ensures that the concentrations of the indicator systems in the multirange indicator are in total sufficiently low to achieve uniform transmission of light through the specimen, and thus a colour change which is attendant in a defined manner on the incident dose.

Another object of the present invention consisted in simplifying the production of a dose-measurement coating, which permits determination of the dose of actinic radiation even within manufacturing plants. According to the invention, this is achieved via use of the multirange indicator described above for the production of a dose-measurement coating for the measurement of UV radiation and/or of electron-beam radiation. A consequence of the advantages described above for this multirange indicator is a particularly simple method of obtaining a coating for the measurement of the incident radiation dose, where it is even possible, because of the external dimensions of the coating—in particular its thickness—to introduce the system into conventional manufacturing plants for web-type products and thus also to achieve reliable process control.

According to another aspect of the present invention, a dose-measurement coating material is proposed for the measurement of UV radiation and/or electron-beam radiation, which provides dose determination for various irradiation wavelengths. The said object was achieved by using a dose-measurement coating material which encompasses the multirange indicator described above. A stable dose-measurement coating material is thus obtained, which can be used to carry out local multirange dose measurements of actinic radiation directly at the surface of three-dimensional products.

The invention should moreover simplify the production of flat dose-measurement elements for the multirange measurement of UV radiation and/or electron-beam radiation. This is achieved via use of the multirange indicator described above. This gives a flat dose-measurement element for stable parallel detection of the dose introduced of actinic radiation from more than a single measurement range.

Another object of the present invention consisted in providing a flat dose-measurement element for the measurement of UV radiation and/or electron-beam radiation, which permits parallel determination of the respective dose of actinic radiation from different wavelength ranges. This object is achieved by a flat dose-measurement element which encompasses a radiation-sensitive layer with the multirange indicator described above. It is particularly advantageous here if the flat dose-measurement element encompasses an outer layer adapted for the defined attenuation of the incident UV radiation and/or electron-beam radiation. A consequence of the use of this type of outer layer is the possibility of reducing, in a defined manner, the intensity of the actinic radiation to which the indicator systems are exposed, thus also permitting the determination of high radiation doses.

It can also be advantageous to provide an outer layer which, at least in essence, completely covers the surface of the external side of the flat dose-measurement element, and which has been manufactured from a material impermeable to gases. This makes it impossible or at least more difficult for gases which distort dose measurement and/or which can adversely affect the long-term stability of the detection result to diffuse to the indicator systems, the result being a further improvement in the reliability of dose determination. Examples of gases of this type can be oxygen or ozone formed during exposure of atmospheric oxygen to actinic radiation (a possible result here being oxidation of the indicator dyes or of free radicals formed as intermediates), other atmospheric gases, such as water vapour (a possible result here being hydrolysis of the constituents of the indicator system), or carbon dioxide (a possible result here, in combination with water, being formation of carbonic acid with adverse results on an indicator system based on acids or on bases), or else other gases and contaminants. The outer layer impermeable to gases can be identical with the attenuating outer layer or different therefrom.

It has moreover proved to be useful if the flat dose-measurement element encompasses an adhesive layer, making it particularly easy, prior to a multirange dose determination, to secure the flat dose-measurement element directly on the surface of a product to be monitored, or to attach it locally within the manufacturing part.

A further improvement in the present invention was achieved in that the flat dose-measurement element encompasses a backing element. The mechanical stability of the flat dose-measurement element is thus increased. The result is not only that it is easier to handle and to secure to the product to be monitored but also that the flat dose-measurement element can be removed intact from the surface of the product once a dose determination has been carried out, without leaving residues of the flat dose-measurement element on the surface of the product. Another advantage of this design is that the flat dose-measurement element can be archived in its original entirety.

A final object of the present invention consisted in providing a method which permits the dose of electromagnetic radiation incident on a test specimen to be determined in parallel for a plurality of wavelength ranges of the radiation. This object is achieved by a method in which at least one portion of the surface of the test specimen is provided with the multirange indicator described above, and the test specimen is then exposed to electromagnetic radiation with wavelengths from the first radiation wavelength range and from the second radiation wavelength range, where the change in the light absorption of the first indicator dye and the change in the light absorption of the second indicator dye are recorded in the visible-light region of the spectrum, and independently of one another, and using calibration values, the dose of the incident electromagnetic radiation with wavelengths from the first radiation wavelength range is determined from the change in the light absorption of the first indicator dye, and the dose of the incident electromagnetic radiation with wavelengths from the second radiation wavelength range is determined from the change in the light absorption of the second indicator dye. When the process according to the invention is carried out, it is in principle possible to obtain, simultaneously and in a simple manner, exact and reliable multirange determination of the radiation dose from different wavelength regions, without the need for any additional steps for this purpose, for example final development or fixing of the indicator dyes, and the multirange detection here can even be carried out in situ. It is thus possible to achieve continuous multirange radiation detection.

Accordingly, the invention provides a multirange indicator with at least two indicator systems. The term indicator is used for each composition which has at least one indicator system and which allows one or more processes of any type to be followed qualitatively or quantitatively, in that this composition provides an optically detectable signal, as a consequence of a chemical reaction, indicating that a system has reached or has moved away from a particular state. The term indicator system is used for any substance or mixture of substances that can be used and that allows a single process to be followed qualitatively or quantitatively, in that this substance or mixture provides an optically detectable signal, as a consequence of a chemical reaction, indicating that a system has reached or has moved away from a particular state.

In the present case, the indicator systems involved are exclusively those for actinic radiation, i.e. by way of example for UV light, gamma radiation or electron-beam radiation, these respectively having certain wavelength ranges. Each of the indicator systems according to the invention here is sensitive to actinic radiation from only one certain wavelength range.

In more precise terms, indicator systems for the purposes of this invention do not merely provide qualitative detection of the presence of the actinic radiation but instead exhibit an alteration whose extent or intensity is proportional to the total amount of incident radiation. The amount of radiation here corresponds to the intensity, integrated over the period of incidence, of the actinic radiation incident in the respective wavelength range, i.e. to the indicator-system-based energy dose of the said radiation.

The indicator according to the invention involves a multirange indicator, i.e. an indicator which can detect actinic radiation from at least two different wavelength ranges, where although these ranges can have some degree of spectral overlap region they are not completely identical. A multirange indicator according to the invention can naturally also have more than two indicator systems, where these provide sensitivity to radiation from a wider range of wavelengths.

The multirange indicator is preferably intended to have been designed as multirange UV indicator. The term multirange UV indicator is used for any indicator which can be used to detect radiation from at least two different wavelength ranges, where at least one of the indicator systems present therein, and indeed preferably all of the indicator systems, is/are sensitive to, and can detect, radiation from the ultraviolet region of the spectrum (approximately from 1 nm to 380 nm).

According to the invention, a multirange indicator with at least two indicator systems sensitive in the UV region can be realized by, for example, adapting the indicator in such a way that the electromagnetic radiation absorbed by the first indicator system and the electromagnetic radiation absorbed by the second indicator system is respectively light from the ultraviolet region of the spectrum. As far as the specific composition of the multirange indicator according to the invention is concerned, this adaptation includes the use, as photolatent Lewis acid or, respectively, photolatent Lewis base of the first indicator system, of a substance whose absorption spectrum in the UV region exhibits a non-zero absorption (corresponding to transmittance which differs from 1.0). As a consequence of absorption of the said UV light, this substance liberates a Lewis acid or, respectively, Lewis base, which causes a first colour change of the first indicator dye. The second indicator dye is moreover then selected in such a way that it undergoes a second colour change as a consequence of UV light absorption by the multirange UV indicator, preferably in that, as second indicator dye, a dye is used whose absorption spectrum in the UV region exhibits a non-zero absorption, and whose structure alters in a chemical reaction as a consequence of absorption of UV light.

The first indicator system of the multirange indicator according to the invention here has a first indicator dye and a photolatent Lewis acid or a photolatent Lewis base.

The photolatent Lewis acid or photolatent Lewis base involves a photochemically active substance, i.e. a substance capable of absorbing energy from incident light in such a way that the said substance is altered in a chemical reaction as a consequence of absorbing the energy, thus liberating a photolatent Lewis acid or photolatent Lewis base. To this end, the photolatent Lewis acid or photolatent Lewis base has a non-zero absorption at the wavelengths of actinic light whose dose is respectively to be monitored, so that the actinic radiation is absorbed completely or at least to some extent by the photolatent

Lewis acid or photolatent Lewis base, and converts the same into an excited energetic state. The consequence of the excited energetic state is the liberation of the Lewis acid or Lewis base. The concentration of free Lewis acid or Lewis base in the multirange indicator is thus locally increased, the result being that the absorption properties of the indicator dye are altered. Since this alteration in the absorption of the indicator dye takes place at wavelengths in the visible region, the alteration of absorption can be followed visually.

The first indicator system comprises, in addition to the photolatent Lewis acid or photolatent Lewis base, a first indicator dye, matched to the photolatent Lewis acid or photolatent Lewis base, i.e. a corresponding indicator dye.

The photolatent Lewis acid or photolatent Lewis base of the first indicator system can in principle be any substance which has a non-zero absorption at least in one wavelength range of the actinic radiation, and which is moreover capable, as a consequence of absorption of the actinic radiation, of liberating a Lewis acid or Lewis base, i.e. producing the same in a chemical reaction or in some other way making it available in the form of free compound, for example in a desorption step or from a Lewis adduct. The Lewis acid or Lewis base can by way of example involve a moiety cleaved from the photolatent Lewis acid or photolatent Lewis base.

Lewis acids are any of the electrophilic electron-pair acceptors, i.e. any of the substances which can undergo addition reactions with electron pairs, examples being molecules and ions having an incomplete noble-gas configuration, i.e. one or more vacant electron positions. For the purposes of this invention, the term Lewis acids also includes in particular Brønsted acids (traditional acids; protic acids), i.e. substances which are, or which comprise, proton donors, and protons themselves are also included within this definition.

Correspondingly, Lewis bases are any of the nucleophilic electron-pair donors, i.e. any of the substances which can provide electron pairs. For the purposes of this invention, the term Lewis bases also in particular includes Brønsted bases (traditional bases) i.e. substances which are, or which comprise, proton acceptors, and hydroxide ions themselves are also included within this definition.

Examples of photolatent Lewis acids that can be used according to the invention are known by way of example from WO 02/101462 A1 and WO 2005/097876 A1, which are expressly incorporated herein by way of reference.

Latent Lewis acids according to WO 2005/097876 A1 are in particular those which are based on a compound of the general formula R¹—CH^{*}R⁰-(A6)R²R³R⁴R⁵—OH. A6 here is an aromatic ring system which has six ring atoms and which optionally can contain a heteroatom or a plurality of heteroatoms and/or further annellated rings. R¹ is selected from the group consisting of hydrogen, alkyl groups (in particular C₁-C₂₀-alkyl groups), alkenyl groups (in particular C₂-C₂₀-alkenyl groups), aryl groups (in particular unsubstituted, or else singly, doubly or triply C₁-C₄-alkyl-substituted phenyl groups), or C₁-C₄-alkoxy groups. R², R³, R⁴, and also R⁵, are selected independently of one another from the group consisting of hydrogen or functional substituents. R⁰ is selected from the group consisting of C₁-C₆-alkyl groups, or groups of the general formula -Z¹-Q¹ or -Z²-Q². Z¹ here is a single bond or a bridging sulphur atom (—S—) or oxygen atom (—O—) or a bridging secondary amine group (—NH—). Q¹ here is a heterocyclic ring system having from 5 to 9 ring atoms, the ring atoms of which can be carbon (C), sulphur (S), oxygen (O) and nitrogen (N), and the ring system here contains at least two, preferably three, particularly preferably at least four, carbon atoms. Q¹ in particular represents morpholine, pyridine (which can optionally have from one to three substituents which are C₁-C₂-alkyl groups or which are hydroxy groups), mercaptobenzoxazole or mercaptobenzothiazole. Z² is a C₁-C₄-alkylene group, which can have substitution by a C₁-C₄-alkyl group or by Q³. Q² and Q³ here are, independently of one another, phenyl groups, which can optionally have from one to three substituent(s), where the substituents are C₁-C₄-alkyl groups, or are hydroxy groups, or are C₅-C₈-cycloalkyl groups, and/or where the substituent is a heterocyclic ring system having from 5 to 9 ring atoms, the ring atoms of which can be carbon (C), sulphur (S), oxygen (O) and nitrogen (N), and the ring system here contains at least two, preferably three, particularly preferably at least four, carbon atoms. It is moreover possible that the hydrogen atom H^{*} bonded to the carbon atom in alpha-position with respect to the substituent R⁰ is cleaved in the form of a proton in a photochemical reaction on exposure to electromagnetic radiation.

Specific examples of photolatent Lewis acids have been described in WO 02/101462 A1, and it is possible, without any restriction to these examples, to use any of these.

The photolatent acids used can also comprise the phenolic antioxidants described in EP 2003/050912. Typical examples of these are compounds from the group of the hydroxyphenylbenzotriazoles, of the hydroxyphenyltriazines or of the hydroxybenzophenones, all of which have a hydroxy group, the arrangement of these being on a phenyl ring in ortho-position in relation to the bond between the phenyl ring and the main skeleton of the molecule.

Examples of photolatent Lewis bases that can be used according to the invention are known for example from EP 0 970 085, WO 03/033500 and WO 2008/009575 A2, expressly incorporated herein by way of reference. It is in particular possible to use the amino compounds covered by the general structure (I) in WO 2008/009575 A2.

In the first indicator system, it is possible that further photolatent Lewis acids or, respectively, photolatent Lewis bases are present in addition to the first photolatent Lewis acid or, respectively, photolatent Lewis base, in order that, integrally with the first indicator system, it is possible to detect actinic radiation from a relatively large wavelength range or from a plurality of mutually separate wavelength ranges.

The Lewis acids or Lewis bases (in particular protons or hydroxide ions) liberated from the photolatent Lewis acids or Lewis bases interact with the first indicator dye and thus cause an alteration of the absorption of the first indicator dye. The first indicator dye must therefore have been designed to be a Lewis-acid-sensitive dye or to be a Lewis-base-sensitive dye, in particular to be an acid-sensitive dye or to be a base-sensitive dye, so as to correspond to the respective photolatent Lewis acids or Lewis bases used. Accordingly, by way of example, any of the known pH-indicator dyes, which alter their colour as a function of proton concentration, can in principle be used as first indicator dye.

However, particularly preferred first indicator dyes are those which have, as atomic structure, a structure derived from fluoran (spiro[isobenzofuran-1,9′-xanthen]-3-one), these being known as “fluorans”. Examples of acid-sensitive fluorans that can be used according to the invention are known for example from WO 2005/097876 A1, which is expressly incorporated herein by way of reference.

Examples of particularly preferred fluorans, without any intention of restriction to this list, are 3-dibutylamino-7-dibenzylaminofluoran, 3-diethylamino-6-methylfluoran, 3-dimethylamino-6-methyl-7-anilinofluoran, 3-diethylamino-6-methyl-7-anilinofluoran, 3-diethylamino-6-methyl-7-(2,4-dimethylanilino)fluoran, 3-diethylamino-6-methyl-7-chlorofluoran, 3-diethylamino-6-methyl-7-(3-trifluoromethylanilino)fluoran, 3-diethylamino-6-methyl-7-(2-chloroanilino)fluoran, 3-diethylamino-6-methyl-7-(4-chloroanilino)fluoran, 3-diethylamino-6-methyl-7-(2-fluoroanilino)fluoran, 3-diethylamino-6-methyl-7-(4-n-octylanilino)fluoran, 3-diethylamino-7-(4-n-octylanilino)fluoran, 3-diethylamino-6-methyl-7-(dibenzylamino)fluoran, 3-diethylamino-7-(dibenzylamino)fluoran, 3-diethylamino-6-chloro-7-methylfluoran, 3-diethylamino-7-tert-butylfluoran, 3-diethylamino-7-carboxyethylfluoran, 3-diethylamino-6-chloro-7-anilinofluoran, 3-diethylamino-6-methyl-7-(3-methylanilino)fluoran, 3-diethylamino-6-methyl-7-(4-methylanilino)fluoran, 3-diethyl-amino-6-ethoxyethyl-7-anilinofluoran, 3-diethylamino-7-methylfluoran, 3-diethylamino-6,8-dimethylfluoran, 3-diethylamino-7-chlorofluoran, 3-diethylamino-7-chlorofluoran, 3-diethylamino-7-(3-trifluoromethylanilino)fluoran, 3-diethylamino-7-(2-chloroanilino)fluoran, 3-diethylamino-7-(2-fluoroanilino)fluoran, 3-diethylaminobenzo[a]fluoran, 3-diethylaminobenzo[c]fluoran, 3-dibutylamino-6-methylfluoran, 3-dibutylamino-6-methyl-7-anilinofluoran, 3-dibutylamino-6-methyl-7-(2,4-dimethylanilino)fluoran, 3-dibutylamino-6-methyl-7-(2-chloroanilino)fluoran, 3-dibutylamino-6-methyl-7-(4-chloroanilino)fluoran, 3-dibutylamino-6-methyl-7-(2-fluoroanilino)fluoran, 3-dibutylamino-6-methyl-7-(3-trifluoromethylanilino)fluoran, 3-dibutylamino-6-ethoxyethyl-7-anilinofluoran, 3-dibutylamino-6-chloro-anilinofluoran, 3-dibutylamino-6-methyl-7-(4-methylanilino)fluoran, 3-dibutyamino-7-(2-chloroanilino)fluoran, 3-dibutylamino-7-(2-fluoroanilino)fluoran, 3-dipentylamino-6-methyl-7-anilinofluoran, 3-dipentylamino-6-methyl-7-(4-2-chloroanilino)fluoran, 3-dipentylamino-7-(3-trifluoromethylanilino)fluoran, 3-dipentylamino-6-chloro-7-anilinofluoran, 3-dipentylamino-7-(4-chloroanilino)fluoran, 3-pyrrolidino-6-methyl-7-anilinofluoran, 3-piperidino-6-methyl-7-anilinofluoran, 3-(N-methyl-N-propylamino)-6-methyl-7-anilinofluoran, 3-(N-methyl-N-cyclohexylamino)-6-methyl-7-anilinofluoran, 3-(N-ethyl-N-cyclohexylamino)-6-methyl-7-anilinofluoran, 3-(N-ethyl-N-n-hexylamino)-7-anilinofluoran, 3-(N-ethyl-p-toluidino)amino-6-methyl-7-anilinofluoran, 3-(N-ethyl-p-toluidino)amino-7-methylfluoran, 3-(N-ethyl-N-isoamylamino)-6-methyl-7-anilinofluoran, 3-(N-ethyl-N-isoamylamino)-7-(2-chloroanilino)fluoran, 3-(N-ethyl-N-isoamylamino)-6-chloro-7-anilinofluoran, 3-(N-ethyl-N-tetrahydrofurfurylamino)-6-methyl-7-anilinofluoran, 3-(N-ethyl-N-isobutylamino)-6-methyl-7-anilinofluoran, 3-(N-butyl-N-isoamylamino)-6-methyl-7-anilinofluoran, 3-(N-isopropyl-N-3-pentylamino)-6-methyl-7-anilinofluoran, 3-(N-ethyl-N-ethoxypropylamino)-6-methyl-7-anilinofluoran, 3-cyclohexylamino-6-chlorofluoran, 2-methyl-6-p-(p-dimethylaminophenyl)-aminoanilinofluoran, 2-methoxy-6-p-(p-dimethylaminophenyl)aminoanilinofluoran, 2-chloro-3-methyl-6-p-(p-phenylaminophenyl)aminoanilinofluoran, 2-diethylamino-6-p-(p-dimethylaminophenyl)aminoanilinofluoran, 2-phenyl-6-methyl-6-p-(p-phenylaminophenyl)-aminoanilinofluoran, 2-benzyl-6-p-(p-phenylaminophenyl)aminoanilinofluoran, 3-methyl-6-p-(p-dimethylaminophenyl)aminoanilinofluoran, 3-diethylamino-6-p-(p-diethylaminophenyl)aminoanilinofluoran, 3-diethylamino-6-p-(p-dibutylaminophenyl)-aminoanilinofluoran, 2,4-dimethyl-6-[(4-dimethylamino)anilino]fluoran, 3-diethylamino-6-methylfluoran, 3-diethylamino-6-methyl-7-anilinofluoran, 3-diethylamino-6-methyl-7-(2,4-dimethylanilino)fluoran, 3-diethylamino-6-methyl-7-chlorofluoran, 3-diethylamino-6-methyl-7-(3-trifluoromethylanilino)fluoran, 3-diethylamino-6-methyl-7-(2-chloroanilino)fluoran, 3-diethylamino-6-methyl-7-(4-chloroanilino)fluoran, 3-diethylamino-6-methyl-7-(2-fluoroanilino)fluoran, 3-diethylamino-7-(4-n-octylanilino)fluoran, 3-diethylamino-7-(dibenzylamino)fluoran, 3-diethylamino-6-chloro-7-methylfluoran, 3-diethylamino-7-tert-butylfluoran, 3-diethylamino-7-carboxyethylfluoran, 3-diethylamino-6-chloro-7-anilinofluoran, 3-diethylamino-6-methyl-7-(3-methylanilino)fluoran, 3-diethylamino-6-methyl-7-(4-methylanilino)fluoran, 3-diethylamino-7-methylfluoran, 3-diethylamino-6,8-dimethylfluoran, 3-diethylamino-7-chlorofluoran, 3-diethylamino-7-(3-trifluoromethylanilino)fluoran, 3-diethylamino-7-(2-chloroanilino)fluoran, 3-diethylamino-7-(2-fluoroanilino)fluoran, 3-diethylaminobenzo[a]fluoran-6-ethoxyethyl-7-anilinofluoran, 3-dibutylamino-6-chloroanilinofluoran, 3-dipentylamino-6-methyl-7-anilinofluoran, 3-(N-methyl-N-propylamino)-6-methyl-7-anilinofluoran, 3-(N-methyl-N-cyclohexylamino)-6-methyl-7-anilinofluoran, 3-(N-ethyl-N-cyclohexylamino)-6-methyl-7-anilinofluoran, 3-(N-ethyl-N-n-hexylamino)-7-anilinofluoran, 3-(N-ethyl-p-toluidino)amino-6-methyl-7-anilinofluoran, 3-(N-ethyl-p-toluidino)amino-7-methylfluoran, 3-(N-ethyl-N-isoamylamino)-6-methyl-7-anilinofluoran, 3-(N-ethyl-N-tetrahydrofurfurylamino)-6-methyl-7-anilinofluoran, 3-(N-ethyl-N-isobutylamino)-6-methyl-7-anilinofluoran, 3-(N-butyl-N-isoamylamino)-6-methyl-7-anilinofluoran, 3-(N-isopropyl-N-3-pentylamino)-6-methyl-7-anilinofluoran, 3-(N-ethyl-N-ethoxypropylamino)-6-methyl-7-anilinofluoran, 3-cyclohexylamino-6-chlorofluoran, 7-(N-ethyl-N-isopentylamino)-3-methyl-1-phenylspiro[4H-chromenopyrazole-4-(1H)-3′-phthalide, 3-diethylamino-6-methyl-7-anilinofluoran, 3-diethylamino-6-methyl-7-(2,4-dimethylanilino)fluoran, 3-diethylamino-6-methyl-7-chlorofluoran, 3-diethylamino-7-(4-n-octylanilino)fluoran, 3-diethylamino-7-(dibenzylamino)fluoran, 3-diethylamino-7-tert-butylfluoran, 3-diethylamino-7-carboxyethylfluoran, 3-diethylamino-6,8-dimethylfluoran, 3-diethylamino-benzo[a]fluoran, 3-diethylamino-benzo[c]fluoran, 3-dibutylamino-6-methyl-7-anilinofluoran, 3-dipentylamino-6-methyl-7-anilinofluoran, 3-(N-methyl-N-propylamino)-6-methyl-7-anilinofluoran, 3-(N-methyl-N-cyclohexylamino)-6-methyl-7-anilinofluoran, 3-(N-ethyl-p-toluidino)amino-7-methylfluoran, 3-(N-ethyl-N-isoamylamino)-6-methyl-7-anilinofluoran, 3-(N-ethyl-N-isobutylamino)-6-methyl-7-anilinofluoran, 3-(N-ethyl-N-ethoxypropylamino)-6-methyl-7-anilinofluoran, 3-cyclohexylamino-6-chlorofluoran and 7-(N-ethyl-N-isopentylamino)-3-methyl-1-phenylspiro[4H-chromenopyrazole-4-(1H)-3′-phthalide.

Commercially available fluorans are marketed by way of example as Pergascript Black T-R, Pergascript Black T-2R, Pergascript Green 1-2GN, Pergascript Orange I-G, Pergascript blue I-2RN, Orange DCF, Red DCF, Orange 100, Red PSD-V, Red ETPM.

It is naturally also possible to use dyes other than fluorans as first indicator dye, for example dyes based on lactones, on benzoxazines, on spiropyrans or on phthalides.

In principle, for the first indicator system it is possible to combine any desired Lewis-acid-sensitive indicator dyes with any desired photolatent Lewis acids, or any desired Lewis-base-sensitive indicator dyes with any desired photolatent Lewis bases. The selection of the specific photolatent Lewis acid to be used or photolatent Lewis base to be used should take into account the respective specific application and in particular here should be appropriate for the wavelengths of the wavelength range to be monitored. The first indicator system can also moreover comprise more than one indicator dye, in order to provide a colour change with particularly high contrast. The dose can be determined here from the total of the individual doses for each of the indicator dyes of the first indicator system.

In the indicator systems to be used according to the invention, it is usual to realize the indicator dye and the photolatent Lewis acid or the photolatent Lewis base in the form of different compounds. In one particular embodiment, however, it is possible that the photolatent Lewis acid or, respectively, photolatent Lewis base and the indicator dye can be realized in the form of a single compound. In a possible example of this type of embodiment, the indicator dye and the photolatent Lewis acid or photolatent Lewis base of the indicator system have been bonded to one another via the polymer skeleton as constituents of a fundamental polymeric skeleton, thus having been copolymerized into the same polymer, for example in the form of copolymers or in a subsequent graft reaction.

The multirange indicator according to the invention has, in addition to the first indicator system, a second indicator system. The second indicator system encompasses a second indicator dye, and is capable of changing the light absorption of the second indicator dye in a second reaction on exposure to electromagnetic radiation with wavelengths from a second radiation wavelength range. The second indicator system can in principle be any of the indicator systems of this type in which the second indicator dye is a triphenylmethane dye in which the central carbon atom of the methane group has bonding to a structural unit selected from the group consisting of halogens, pseudohalogens, chalcogens, sulphates and substituted or unsubstituted tosylates, and in which at least one of the C₆ rings of the phenyl groups has, in ortho- or para-position with respect to the bond to the central methyl carbon atom, an electron-withdrawing substituent with +M effect, and/or has, in meta-position with respect to the bond to the central methyl carbon atom, an electron-donating substituent with +M effect.

Triphenylmethane dye (triarylmethane dye) is any of the dyes having an atomic structure derived from triphenylmethane [(H₅C₆)₃C—H]. According to the invention, the methyl group of the triphenylmethane dye has a further substituent in addition to the phenyl groups, and among the known triphenylmethane dyes it is therefore possible to use only reduced forms, for example those dyes which derive from the triaryl carbinoles, examples being rosaniline [(4-amino-3-methylphenyl)bis(4-aminophenyl)methanol] or pararosaniline [tris(4-aminophenyl)methanol] or else which derive from the leuco bases of the oxidized dyes, e.g. the leuco bases of Doebner's violet, malachite green, Brilliant Green, Patent Blue VF, fuchsine, parafuchsine, acid fuchsin, new fuchsin, Hoffmann's violet, methyl violet, crystal violet, methyl blue, methyl green, aniline blue, water blue, alkali blue or aurine.

According to the invention, at least one of the three phenyl groups is in substituted form, and this group may have one or more substituents. However, it is preferable that more than one phenyl group is present in substituted form, and this therefore means two phenyl groups (as is the case for example with the leuco bases of the following diaminotriarylmethane dyes: Doebner's violet, malachite green, Brilliant Green, Patent Blue VF), or all three of the phenyl groups (as is the case for example with rosaniline or pararosaniline or with the leuco bases of fuchsine, parafuchsine, acid fuchsin, new fuchsin, Hoffmann's violet, methyl violet, crystal violet, methyl blue, methyl green, aniline blue, water blue, alkali blue or aurine). In each case here it is possible that the three phenyl groups of the triphenylmethane dye are identical (as is the case for example with pararosaniline) or are different (as is the case for example with rosaniline).

According to the invention, a substituent of the C₆ ring of a phenyl group can be any of those substituents which have +M effect (mesomeric effect of “+” type). Substituents which have a mesomeric effect participate in the mesomerism of an aromatic ring system bonded to the said groups, and these substituents therefore enlarge the mesomeric system. Substituents exhibiting +M effect generally have at least one free electron pair which can be made available for mesomerism. The result is to increase the total electron density of the mesomeric system. Examples of typical substituents having +M effect are, without any restriction to this list: unsubstituted or substituted aryl groups, amino groups (—NH₂), amine groups (mono and di; —NHR and, respectively, —NR₂), hydroxy groups (protonated and deprotonated; —OH and, respectively, —O⁻), ether groups (—OR), ester groups (—OCOR), amide groups (—NHCOR), acrylic acid groups (—CH═CH—COOH) or halogens, such as fluorine (—F), chlorine (—Cl), bromine (—Br) or iodine (—I), where R is respectively any desired unsubstituted or substituted organic moiety.

If the substituent with +M effect is in ortho-position or in para-position with respect to the bond of the C₆ ring of the phenyl group to the central methyl carbon atom, according to the invention it is additionally necessary that an electron-withdrawing substituent is involved. In contrast, if the substituent is in meta-position, according to the invention it is additionally necessary that an electron-donating substituent is involved. The term electron-donating and, respectively, electron-withdrawing is used for substituents which have positive and, respectively, negative inductive effect (+I effect and, respectively, −I effect). An inductive effect arises when electronegativity differences between atoms or functional groups of a molecule polarize a sigma-electron-pair bond situated between these.

In the case of positive inductive effect, a relatively electropositive substituent displaces the electron-pair bond away from itself, thus increasing electron density at the adjacent relatively electronegative atomic structure. By way of example, this occurs if the substituent has a negative charge or has low electronegativity or is present in hybridized form (i.e. having hybrid orbitals). Examples of typical electron donating substituents are—without any restriction caused by this list—tert-butyl groups (—C(CH₃)₃), isopropyl groups (—CH(CH₃)₂), ethyl groups (—C₂H₅), methyl groups (—CH₃), deprotonated hydroxy groups (—O⁻) or alkyl groups (—R).

In contrast to this, in the case of negative inductive effects, a relatively electronegative substituent attracts the electron-pair bond relatively strongly towards itself, thus reducing electron density at the adjacent relatively electropositive atomic structure. By way of example, this occurs when the substituent has a positive charge or has high electronegativity. Examples of typical electron-withdrawing substituents are—without any restriction caused by this list—protonated hydroxy groups (—OH), carboxy groups (—COOH), nitro groups (—NO₂), amino groups (—NH₂), phenyl groups (—C₆H₅) or halogens, such as fluorine (—F), chlorine (—Cl), bromine (—Br) or iodine (—I). The hydrogen atom (—H) exhibits no inductive effect.

A final requirement of the invention is that the second indicator dye is a triphenylmethane dye which has, directly bonded at its central carbon atom of the methane group, a structural unit which can easily be cleaved from the triphenylmethane dye in a photochemical reaction. To this end, the structural unit is selected from the group consisting of halogens (such as fluorine (—F), chlorine (—Cl), bromine (—Br) or iodine (—I)), pseudohalogens (e.g. nitrile groups (—CN), azide groups (—N₃), cyanate groups (—OCN), isocyanate groups (—NCO), nitrile oxide groups (—CNO), thiocyanate groups (—SCN), thioisocyanate groups (—NCS) or selenocyanate groups (—SeCN)), substituted or unsubstituted tosylates (which derive from toluenesulphonic acid —O—SO₂—C₆H₄—CH₃), sulphate (—O—SO₂—O⁻ and its derivatives hydrogensulphate —O—SO₂—OH and sulphuric ester groups —O—SO₂—OR, in which the sulphate likewise has direct bonding to the central carbon atom of the methane group of the dye, where R is respectively any desired unsubstituted or substituted organic moiety), and also chalcogens (e.g. oxygen, sulphur or selenium, where these bond the central carbon atom of the methane group of the triphenylmethane dye to a further moiety, examples therefore being hydroxy groups (—OH), ether groups (—OR), ester groups (—O—CO—R), thiol groups (—SH), thioether groups (—SR), thioester groups (—S—CO—R)), without any restriction deriving from the above lists of specific examples.

In principle, the second indicator dye used can comprise any desired compound which meets the above preconditions. The practical selection of a specific second indicator dye here should take into account the respective specific application and particularly here be appropriate for the wavelengths of the wavelength ranges to be monitored, and secondly be appropriate for the composition of the first indicator system.

A preferred example that may be mentioned and that can be used according to the invention is pararosaniline nitrile, in which the three phenyl groups respectively have amino groups as electron-withdrawing substituents in para-position with respect to the bond to the central methyl carbon atom, and in which the structural unit bonded to the central carbon atom of the methane group is a cyano group (nitrile group). Pararosaniline nitrile dye is transparent prior to irradiation and absorbs electromagnetic radiation from the UVB and UVC region. The photochemical reaction that proceeds here causes an increase in absorption in the visible region of the spectrum (with a maximum at about 550 nm), and the colour of the dye thus becomes red.

Dose-measurement systems based on leuco cyanides of pararosaniline (pararosaniline nitrile) or on derivatives thereof have been previously disclosed in McLaughlin et al.: “Radiochromic Plastic Films for Accurate Measurement of Radiation Absorbed Dose and Dose Distribution” Radiat. Phys. Chem. Vol. 10, 119-125 (1977), and also McLaughlin et al.: “The Gamma-Ray Response of Radiochromic Dye Films at Different Absorbed Dose Rates” Radiat. Phys. Chem. Vol. 18 No. 5-6, 987-999 (1981), Rativanich et al.: “Liquid Radiochromic Dosimetry” Radiat. Phys. Chem. Vol. 18 No. 5-6, 1001-1010 (1981), Uribe et al.: “Possible Use of Electron Spin Resonance of Polymer Films Containing Leukodyes for Dosimetry” Radiat. Phys. Chem. Vol. 18 No. 5-6, 1011-1016 (1981), Buenfil-Burgos et al.: “Thin Plastic Radiochromic Dye Films as Ionizing Radiation Dosimeters” Radiat. Phys. Chem. Vol. 22 No. 3-5, 325-332 (1983) or Schönbacher et al.: “Colour Dosemeters for High Level Radiation Dosimetry”, Radiation Protection Dosimetry, Vol. 34, No. 1/4, 311-314 (1990).

Applications of pararosaniline nitrile as indicator dye are described in the prior art, for example in DE 10 2004 022 071 A1, which is hereby incorporated by way of reference with respect to systems that can be used for the purposes of the present invention. DE 10 2004 022 071 describes a dose-measurement film for electron-beam radiation. This involves a composite composed of a radiation-sensitive layer of thickness 20 μm on a polyester film of thickness 50 μm, where the radiation-sensitive layer in essence consists of a polyester coating material and a leuco cyanide of pararosaniline (pararosaniline nitrile) as radiation-sensitive dye. To produce the measurement film, the dissolved constituents of the layer are applied to the polyester film and the solvent is removed in a drying stage.

The multirange indicator according to the invention therefore comprises a first indicator system and a second indicator system. In the simplest case, the multirange indicator has no further constituents beyond these. However, the multirange indicator can naturally also comprise further constituents, examples being further indicator systems or auxiliaries.

To realize the multirange UV indicator here, the first indicator system can be capable of absorbing light from the UVA region of the spectrum, and the second indicator system can be capable of absorbing light from the UVB and/or UVC region of the spectrum. This includes the use, as photolatent Lewis acid or, respectively, Lewis base of the first indicator system, of a substance which absorbs light from the UVA region of the spectrum and therefore has a non-zero absorption in that region, while the second indicator dye of the second indicator system absorbs light from the UVB region of the spectrum, light from the UVC region of the spectrum, or light not only from the UVB region but also from the UVC region of the spectrum.

With respect to easier handling of the multirange indicator, it is moreover desirable that the transition of the indicator system for the UVA region is spectrally narrow, so that this indicator system has at most low absorption in the blue region of the visible-light spectrum (i.e. at wavelengths above 450 nm or preferably above 400 nm), so as to avoid a colour-change reaction triggered simply by absorption of daylight. Substances that can be used according to the invention with this type of absorption are known from the prior art for the UVA region, the UVB region and the UVC region.

The colour changes occurring with the first indicator dye and with the second indicator dye can be as desired; in particular, it is possible to select the colours of the dyes prior to irradiation with actinic radiation, and also after irradiation, in any desired suitable manner so that the colour changes of the two indicator systems are detectable independently of one another and are moreover easily discernible on the respective appropriate background. In particular, the first indicator dye and/or the second indicator dye can take the form of leuco dye prior to exposure to the electromagnetic radiation. Leuco dye (leuco compound) is the term used for any compound which is the precursor of a substance which absorbs light from the visible-light region (a dye), where the precursor itself is colourless and therefore has no, or at most low, absorption in the visible-light region. A leuco dye can usually be converted into the corresponding coloured dye by a redox reaction, and frequently takes the form of (mostly reduced) leuco base of the said dye.

The consistency of the substances in the multirange indicator can be as desired. By way of example, the multirange indicator can take the form of a liquid solution of the indicator systems or else can take the form of a solid, for example in the form of a pulverulent indicator. The multirange indicator preferably takes the form of a constituent of a coating material which can be solidified (which comprises, for example, in addition to the multirange indicator, a liquid or solid binder and also, if appropriate, further additives) or of a layer (for example of a self-supporting film, or of a coating or the like). For application, the indicator systems are usually embedded into a solid or semisolid matrix material, and this matrix material can be selected as desired, as long as the material itself has no absorption (or at least a defined absorption) in the region of the detection wavelengths and of the indicator wavelengths, and does not distort the colour-change reaction (i.e. for example does not interact with the Lewis acid or Lewis base liberated) and moreover preferably is not photochemically reactive, so that it does not adversely affect any of the colour-change processes.

It is moreover possible here to select the matrix material in such a way that the latent Lewis acid or, respectively, latent Lewis base and/or the indicator dyes have little freedom of movement within the matrix, for example by selecting bulky photolatent Lewis acids/bases, indicator dyes and/or matrix polymers, or by using photolatent Lewis acid/bases and/or indicator dyes which have chemical bonding to the matrix. Immobilized indicator systems are thus obtained, in which a change in a Lewis acid or, respectively, Lewis base liberated (which may have some freedom of movement) is recorded locally with spatial resolution, thus permitting a reduction in the level of disruptive interaction with other indicator systems.

The molar amounts of the indicator systems present in this multirange indicator can be any desired suitable amounts which permit detection of the respective actinic radiation dose. In particular, the amount present of the first indicator system and/or of the second indicator system here, in the multirange indicator, can be at least 0.01% by weight and at most 10% by weight.

According to the invention, the multirange indicator can be used to produce a dose-measurement coating for the measurement of UV radiation and/or electron-beam radiation. A dose-measurement coating is considered to be any of the at least in essence layer-type arrangements on the surface of a body which comprise the multirange indicator and which serve for the qualitative or quantitative determination of a dose of actinic radiation incident on the surface of the coating. Coatings of this type can be of any desired shape here, for example take the form of a film extending in two dimensions, or the form of a system covering the three-dimensional surface of a moulding. In particular, the multirange indicator can have been designed as a constituent of a dose-measurement coating material for the measurement of UV radiation and/or electron-beam radiation, where the coating of this coating material can be obtained via application of the dose-measurement coating material to the surface of a body and subsequent hardening or drying.

However, instead of this, the multirange indicator can also be used in any of the other suitable application forms, for example as flat dose-measurement element for the measurement of UV radiation and/or electron-beam radiation. A flat dose-measurement element is any conventional and suitable structure which is in essence flat and which is capable of qualitative and/or quantitative determination of the dose of radiation incident on the flat element. Flat elements of this type can have various shapes, and in particular can be flexible or take the form of a foil, tape, label, film strip or shaped diecut. This type of flat element advantageously has in addition to the multirange indicator on one of its lateral surfaces an adhesive layer which is formed from a self-adhesive mass, i.e. from a pressure-sensitive-adhesive mass or from an adhesive mass which has heat-activated-adhesive properties, thus permitting simple adhesive bonding of the flat element, for example on the surface of the product to be irradiated, on a test specimen, or else in the interior of, or outside, an irradiation apparatus.

The flat dose-measurement element can have a backing element in addition to the adhesive layer. A backing element is the term used for any structural element that is mostly in essence of flat shape, and which performs the function of a permanent backing within the flat dose-measurement element. Any of the suitable backing materials can be used as backing element, examples being foils composed of metal and/or of plastics, flat textile elements (such as wovens, scrims, knits, non-wovens) or combinations composed of such materials, and the design of this backing element can be coherent across the entire surface or interrupted. However, instead of this, the flat dose-measurement element can have been designed without any backing. In principle, a flat element according to the invention, for dose measurement, can therefore have any desired suitable internal structure.

For the purposes of this invention, furthermore, a flat element can also additionally encompass an outer layer (outer element), adapted for the defined attenuation of the incident UV radiation and/or electron-beam radiation. An outer layer here is any sheet-like layer which at least to some extent covers the upper side of the flat element. This layer has been adapted for defined attenuation of the incident UV radiation and/or electron-beam radiation. Adaptation of this type can for example consist in selection of the material of the said layer (for example of the polymer matrix or of an additive present in the layer) in such a way that the material itself absorbs the radiation in at least one of the wavelength ranges of the actinic radiation.

Because the said outer layer has been arranged in front of the indicator systems in the direction of incidence of the actinic radiation (in the optical path), only a defined portion of the incident actinic radiation reaches the indicator systems (filter action with attenuation of the radiation), where it brings about a colour change. It is therefore possible, through selection of outer layers with suitable optical density (absorption) at the respective detection wavelengths, to adapt the available measurement range in a controlled manner to the radiation dose specifically expected. The material of the outer layer can, furthermore, be selected in such a way as to reduce, or indeed suppress, diffusion of gaseous constituents in the environment of the flat element towards the indicator systems or away from the indicator systems (for example by using, as outer layer, an outer foil which is not permeable to oxygen). Since there are clear advantages in the use of an outer layer, in particular with regard to the second indicator system, the outer layer used can, for example, comprise a layer which itself has an absorption in the UVB region and/or UVC region of the spectrum. Reference is made to the prior art described in DE 10 2004 022 071 A1 in connection with the use of an outer layer.

Use of the multirange indicator according to the invention permits determination of the dose of electromagnetic radiation incident on a test specimen, for example as follows: first, at least one portion of the surface of the test specimen is provided with a multirange indicator. If the multirange indicator is used in the form of dose-measurement coating material, the dose-measurement coating material is applied to the test specimen for this purpose. If a flat dose-measurement element is used which comprises the multirange indicator, the flat dose-measurement element can, in contrast, be attached on a subregion of the surface of the test specimen, for example by means of an adhesive bond. It may be necessary to take care here that the subregion of the test specimen which is intended to be exposed to the actinic radiation for the purposes of the irradiation process to be monitored has not been covered by the flat dose-measurement element or by the dose-measurement coating material.

The test specimen is then exposed to electromagnetic radiation with wavelengths from the first radiation wavelength range and from the second radiation wavelength range. A consequence of this exposure to radiation is, within the multirange indicator, in the visible-light region of the spectrum, a change in the absorption of the first indicator dye and also a change in the absorption of the second indicator dye, where the degree of colour change depends on the incident dose in the particular spectral regions (and therefore on the irradiation time and on the irradiation intensity, i.e. energy density).

The respective absorption change in the first indicator dye and that in the second indicator dye are recorded in order to determine the dose. In principle, the recording can take place visually (for example using tables for comparison of chromaticity coordinates) or with use of an apparatus, an example of the latter being spectral resolution using a spectrometer (spectrophotometer, photometer) or a structure composed of a light source whose intensity is constant over time, and of a light detector and also, if appropriate, of optical filters. A transmission arrangement or a reflection arrangement can be used for these measurements. The overall method of recording here can take the form of continuous monitoring of the radiation dose or else can take the form of spot checks at predetermined measurement junctures.

It is advantageous to use two indicator wavelengths for a determination, namely an observation wavelength from each of the wavelength ranges. If the absorption of the first indicator dye and that of the second indicator dye have spectral overlap, a purely visual evaluation is generally disadvantageous, since it then becomes impossible to achieve quantitative determination here. In this case, preference is given to use of an apparatus for determination and evaluation of the absorption, particularly if a computer-assisted method is used here to adapt parameters appropriately for the absorption spectra or transmission spectra.

The resultant absorption values or transmission values can be converted by calculation to the respective incident radiation dose, and it is possible here to take into account the stoichiometry of the photochemical reaction and of the colour-change reaction and to take into account any radiation losses (for example due to diffuse scattering, reflection, or optical filters, for example an outer layer). The latter corrections can by way of example also be achieved by using calibration values. It is thus possible to determine the dose of incident electromagnetic radiation with wavelengths from the first radiation wavelength range, from the change in the light absorption of the first indicator dye, and, in parallel with this, to determine the dose of the incident electromagnetic radiation with wavelengths from the second radiation wavelength range, from the change in the light absorption of the second indicator dye, the two doses being determined independently of one another.

Further advantages and possible applications are apparent from the inventive example below, which has been selected exclusively by way of example to illustrate the inventive concept, without any intention that the specific selection of this example restrict the scope of protection.

To produce a flat dose-measurement element, an ethanolic multirange indicator solution was first produced. This comprised, as matrix polymer, 97% by weight of polyvinyl butyral (Pioloform from Wacker Chemie), and, as acid-sensitive dye of the first indicator system, 1% by weight of a diaminofluoran compound (PERGASCRIPT Green from Ciba Specialty Chemicals), and, as photolatent acid of the first indicator system, 1% by weight of 1,1,3-tris(2-methyl-4-hydroxy-5-tert-butylphenyl)butane (Lowinox CA22 from Great Lakes Corp.), and also, as indicator dye of the second indicator system, 1% by weight of pararosaniline nitrile.

A doctor wire was used to spread the solution on a polyester foil, as permanent backing, and the solution was dried. The thickness of the dried layer on the polyester film was 20 μm.

The resultant flat dose-measurement element was exposed in a laboratory irradiation system (from the company Eltosch) to the radiation from a medium-pressure mercury lamp (180 W/cm), and to this end the flat dose-measurement element was conducted at a constant web speed past the radiation source. Lamp power and web speed were varied here in order to realize different radiation doses. Systems of this type are in practice usually used together with a filter foil as outer layer. Since the demonstration of the principle according to the invention in the inventive example should be kept as simple as possible, the radiation from the lamp used here was only of low intensity, and it was therefore possible to omit any additional filter foils.

To calibrate the resultant flat dose-measurement element, the change in the optical transmittance of the irradiated flat dose-measurement element was determined at an observation wavelength of 520 nm for the first indicator system (colour change of fluoran) and at an observation wavelength of 620 nm for the second indicator system (colour change of pararosaniline nitrile), for the respective radiation dose. In parallel with this, the absolute dose of the UV radiation at the location of the specimen was measured using an electronic measurement device (“Power Puck” from EIT). The said measurement device permitted subdivision into three different measurement ranges (UVC: 250-260 nm; UVB: 280-320 nm; UVA: 320-390 nm).

In order to check the measured values, the correlation between the respective transmittance change measured and the actual radiation dose determined with the aid of the electronic measurement device was determined. For this purpose, for in each case one wavelength range, the two data sets were subjected to multiple linear regression using a quadratic model in the form of A=x₀+x₁ B+x₂B², where A is the radiation dose measured by the electronic measurement device, B is the optical transmittance at the respective wavelength measured (520 nm or 620 nm) and x₀, x₁, and x₂ are the adjustable model parameters.

These studies showed that the transmittance measured for an indication wavelength of 520 nm correlates with a coefficient of determination of 0.92 with the incident dose in the UVA region, while the transmittance measured for an indicator wavelength of 620 nm correlates with a coefficient of determination of 0.90 with the total incident dose in the UVB region and UVC region. This result shows that, using the selected example of the flat dose-measurement element according to the invention, it is possible to determine differentiated and therefore wavelength-range-specific dose information corresponding to the different wavelength ranges.

Furthermore, it was found that the dose information thus calculated from the transmittance change had at most low dependency on the respective lamp power and on the selected throughput speed, and is therefore almost entirely independent of the incident radiation dose per unit of time, thus permitting non-linear processes to be neglected here.

To determine the stability of the irradiated flat dose-measurement elements, these were stored under defined climatic conditions at a temperature of 23° C. and at relative humidity of 50% for 24 hours, and transmittance was recorded at the two indicator wavelengths. No colour change was observed here. The flat dose-measurement elements according to the invention therefore also have high stability.

To determine the storage stability of the unirradiated dose-measurement elements, these were similarly stored under defined climatic conditions at a temperature of 23° C. and relative humidity of 50% for 24 hours, and here the flat dose-measurement elements were stored with exposure to daylight (at a distance of 2 m behind window glass, but without direct insolation). No colour change was observed in the specimens here, and the conclusion is therefore similarly high storage stability.

The experiments therefore confirm the excellent suitability of the multirange indicator according to the invention in a flat dose-measurement element for the parallel detection of actinic radiation in different wavelength ranges.

1. Multirange indicator with a first indicator system, which encompasses a first indicator dye and encompasses a photolatent Lewis acid or photolatent Lewis base, and which, on exposure of the photolatent Lewis acid or, respectively, photolatent Lewis base to electromagnetic radiation with wavelengths from a first radiation wavelength range, is capable of liberating a Lewis acid or, respectively, Lewis base and of changing the light absorption of the first indicator dye during a first reaction of the first indicator dye with the liberated Lewis acid or, respectively, Lewis base,
wherein,
the multirange indicator has, in addition to the first indicator system, a second indicator system, which encompasses a second indicator dye, and which is capable of changing the light absorption of the second indicator dye in a second reaction on exposure to electromagnetic radiation with wavelengths from a second radiation wavelength range, where
the second indicator dye is a triphenylmethane dye, in which the central carbon atom of the methane group has bonding to a structural unit selected from the group consisting of halogens, pseudohalogens, chalcogens, sulphates and substituted or unsubstituted tosylates, and in which
at least one of the C₆ rings of the phenyl groups has, in ortho- or para-position with respect to the bond to the central methyl carbon atom, an electron-withdrawing substituent with +M effect, and/or has, in meta-position with respect to the bond to the central methyl carbon atom, an electron-donating substituent with +M effect.

2. Multirange indicator according to claim 1, which has been designed as multirange UV indicator and has been adapted in such a way that the electromagnetic radiation absorbed by the first indicator system and the electromagnetic radiation absorbed by the second indicator system is respectively light from the ultraviolet region of the spectrum.

3. Multirange indicator according to claim 2, wherein one of the two indicator systems is capable of absorbing light from the UVA region of the spectrum and the other of the two indicator systems is capable of absorbing light from the UVB region of the spectrum and/or from the UVC region of the spectrum.

4. Multirange indicator according to claim 1, wherein the first indicator dye and/or the second indicator dye takes the form of leuco dye prior to exposure to the electromagnetic radiation.

5. Multirange indicator according to claim 1, wherein the photolatent Lewis acid of the first indicator system encompasses at least one photolatent Brønsted acid, and the first indicator dye of the first indicator system encompasses at least one Brønsted-acid-sensitive dye.

6. Multirange indicator according to claim 5, wherein the acid-sensitive dye has been selected from the group consisting of the fluorans.

7. Multirange indicator according to claim 1, wherein the structural unit bonded to the central carbon atom of the methane group of the triphenylmethane dye is a cyano group.

8. Multirange indicator according to claim 7, wherein the triphenylmethane dye is pararosaniline nitrile.

9. Multirange indicator according to claim 1, wherein the amount present of the first indicator system and/or of the second indicator system is at least 0.01% by weight and at most 10% by weight.

10. A method of producing a dose measurement coating for the measurement of UV radiation and/or of electron-beam radiation comprising employing a multirange indicator according to claim 1.

11. Dose-measurement coating material for the measurement of UV radiation and/or electron-beam radiation, encompassing a multirange indicator according to claim 1.

12. A method for producing a flat dose-measurement element for the measurement of UV radiation and/or electron-beam radiation comprising employing a multirange indicator according to claim 1.

13. Flat dose-measurement element for the measurement of UV radiation and/or electron-beam radiation, encompassing a radiation-sensitive layer with a multirange indicator according to claim 1.

14. Flat dose-measurement element according to claim 13, wherein the flat dose-measurement element encompasses an outer layer, which has been adapted for the defined attenuation of the incident UV radiation and/or electron-beam radiation.

15. Flat dose-measurement element according to claim 13, wherein the flat dose-measurement element encompasses an adhesive layer.

16. Flat dose-measurement element according to claim 13, wherein the flat dose-measurement element encompasses a backing element.

17. Method for the determination of the dose of electromagnetic radiation incident on a test specimen, encompassing the following steps:
providing at least one portion of the surface of the test specimen with a multirange indicator according to claim 1,
exposing the test specimen to electromagnetic radiation with wavelengths from the first radiation wavelength range and from the second radiation wavelength range,
recording the change in the light absorption of the first indicator dye and the change in the light absorption of the second indicator dye in the visible-light region of the spectrum, and
determining, independently of one another, and using calibration values, the dose of the incident electromagnetic radiation with wavelengths from the first radiation wavelength range from the change in the light absorption of the first indicator dye, and determining the dose of the incident electromagnetic radiation with wavelengths from the second radiation wavelength range from the change in the light absorption of the second indicator dye.
